(19) United States
(12) Patent Application Publication
(10) Pub. No.: US 2026/0162250 A1
(43) Pub. Date: Jun. 11, 2026

(54) SYSTEMS AND METHODS FOR QUANTIFYING CHANGE IN ONE OR MORE PATHOLOGY IN THE RETINAL COLOR FUNDUS IMAGE

(71) Applicant: iHealthScreen Inc., Queens Village, NY (US)

(72) Inventor: Alauddin BHUIYAN, Queens Village, NY (US)

(21) Appl. No.: 18/971,640

(22) Filed: Dec. 6, 2024

(51) Int. Cl. G06T 7/00; G06T 7/10; G16H 15/00; G16H 50/20; G16H 50/30

(52) U.S. Cl. CPC G06T 7/0012; G06T 7/10; G16H 15/00; G16H 50/20; G16H 50/30; G06T 2207/30041; G06T 2207/30096; G06T 2207/30101

(57) ABSTRACT

A method for detecting one or more pathology or an indication of one or more pathology, disease or condition progression is described. The method includes registering two or more retinal images, the two or more images including images obtained at differing times; detecting one or more pathology or indication of one or more pathology, disease or condition in the registered two or more images; locating the detected one or more pathology or indication of one or more pathology, disease or condition in a plurality of the two or more images; and quantifying the one or more pathology or indication of the one or more pathology or indication in a plurality of the two or more images to thereby detect the one or more pathology or indication of the one or more pathology, disease or condition progression.

### TECHNICAL FIELD

The present application relates to systems and methods for detecting one or more pathology or an indication of one or more pathology, disease, or condition progression. More particularly, the application relates to systems and methods for quantifying change in one or more drusen and/or one or more bright lesions in the retina.

### BACKGROUND

Early detection of a disease or condition is desirable for many reasons including treatment, outcome and cost. This is particularly true of ocular diseases and ocular conditions because the loss of sight is undesirable.

Conventional ocular examinations include routine examination by medical optometrists, ophthalmologists, orthoptists and other medical and/or health practitioners. The retinal vascular network and pathologies such as drusen, geographic atrophy, and exudates have been indicated as useful factors in the detection of disease and conditions.

Conventional analysis of the retinal vascular network includes manual examination, qualitative analysis, and semiautomatic methods, which are time consuming, costly, and prone to inconsistencies and/or human error. For example, the manual or semi-automatic measurement values vary from one inspection to the next, even when the same grader is involved.

Accordingly, there is a need for alternative methods of analyzing the retina and quantifying changes in the retina.

### SUMMARY

The present disclosure is broadly directed to a method and system for detecting one or more pathology or an indication of one or more pathology, disease or condition progression. In one particular embodiment, the disclosure relates to a method and system for quantifying change in one or more drusen and/or one or more bright lesions in the retina.

In a first aspect, the present disclosure provides a method for detecting one or more pathology or an indication of one or more pathology, disease or condition progression, the method including: registering two or more retinal images, the two or more images including images obtained at differing times; detecting one or more pathology or indication of one or more pathology, disease or condition in the registered two or more images; locating the detected one or more pathology or indication of one or more pathology, disease or condition in a plurality of the two or more images; and quantifying the one or more pathology or indication of the one or more pathology or indication in a plurality of the two or more images to thereby detect the one or more pathology or indication of the one or more pathology, disease or condition progression.

In a second aspect, the present disclosure provides a system for detecting one or more pathology or an indication of one or more pathology, disease or condition progression, the system including: a processor to: register two or more retinal images, the two or more images including images obtained at differing times; detect one or more pathology or indication of one or more pathology, disease or condition in the registered two or more images; locate the detected one or more pathology or indication of one or more pathology, disease or condition in a plurality of the two or more images; and quantify the one or more pathology or indication of the one or more pathology or indication in a plurality of the two or more images to thereby detect the one or more pathology or indication of the one or more pathology, disease or condition progression.

In a third aspect, the present disclosure provides a computer program product for detecting one or more pathology or an indication of one or more pathology, disease or condition progression, the computer program product including: a computer usable medium and computer readable program code embodied on said computer usable medium for displaying data, the computer readable code including: computer readable program code devices (i) configured to cause the computer to register two or more retinal images, the two or more images including images obtained at differing times; computer readable program code devices (ii) configured to cause the computer to detect one or more pathology or indication of one or more pathology, disease or condition in the registered two or more images; computer readable program code devices (iii) configured to cause the computer to locate the detected one or more pathology or indication of one or more pathology, disease or condition in a plurality of the two or more images; and computer readable program code devices (iv) configured to cause the computer to quantify the one or more pathology or indication of the one or more pathology or indication in a plurality of the two or more images to thereby detect the one or more pathology or indication of the one or more pathology, disease or condition progression.

According to any one of the above aspects, the disclosure may further include providing one or more parameters of the quantified one or more pathology or indication of one or more pathology, disease or condition.

The registering may include an automatic registration or a semi-automatic registration. The automatic registration may include one or more algorithm for biomedical image segmentation. The one or more algorithm may include a U-net algorithm.

The semi-automatic registration may include one or more user directed selection. The user directed selection may include a point or region of interest. The point or region or interest may include one or more pixels which correspond to a branch point. The one or more pixels may be selected by a user who clicks or otherwise selects the one or more pixels as the point or region of interest. The semi-automatic registration may further include cropping a region at the clicked or otherwise selected point. The region cropped may include 50×50; 100×100; or 200×200 pixels. The semi-automatic registration may further include vessel segmentation. The vessel segmentation may include using an intensity ratio between an average filtered image with a window and a Gaussian filter image with large standard deviation. The window may include 10×10; 20×20; or 50×50. The semi-automatic registration may further include determining a nearest branch point from the reference click or otherwise selected point or region. The nearest branch point may be stored in a list.

According to any one of the above aspects, the image registration may include one or more of retinal blood vessel segmentation; vessel centerline computation; detecting and extracting parameters of the branch points of the vessels; finding potential matched branch point lists among the two or more images; determining a best registration method; and applying a transformation matrix.

The best registration method may include one or more of: rigid; affine; projective; and quadratic. The best registration method may be selected based on one or more matched branch points and success or rejection of one or more registration method.

The one or more pathology or indication of one or more pathology, disease or condition in the registered two or more images may include one or more drusen and/or one or more bright lesions in the retina.

According to any one of the above aspects, the one or more drusen and/or one or more bright lesions may be detected using a segmentation and quantification method. The segmentation and quantification method may include one or more of intensity profiling; boundary edge detection; shape analysis; vessel segmentation; optic disc detection; and optic disc removal. Two or more segmentation and quantification methods may be used to detect the one or more drusen and/or the one or more bright lesions and they may be combined to produce a final output. The first segmentation and quantification method may include an intensity ratio between surrounding pixels. The second segmentation and quantification method may include finding one or more regions based on boundary edge pixels and an intensity ratio between neighbor pixels and the potential area around a seed point, then merging these regions, defining all of them as a drusen or bright lesion area, and quantifying the total number of pixels. The second segmentation and quantification method may include performing shape analysis using an intensity ratio between neighbor pixels and the potential seed points. The drusen or bright lesion area may be segmented by merging the intensity and edge-based region identification and the total number of pixels may be quantified.

The registration may include identifying a same or identical reference location of a retina in a plurality of the two or more by images.

According to any one of the above aspects, a same pathological feature or indication of pathology, disease or condition may be found on a plurality of the two or more images. The same pathological feature or indication or pathology, disease or condition may be found through a seed point of the corresponding pathological feature or indication of pathology, disease or condition. Overlapping regions within the registered two or more images and the area changes may be determined by computing a change in an area of pathology or indication or one or more pathology, disease or condition. One or more parameters of the area of newly appeared, disappeared and steady pathological feature or indication or pathology, disease or condition between the images may be extracted.

According to any one of the above aspects, after registration and detection, changes from a plurality of the two or more segmented images may be determined in a common shared area. The common shared area may be an overlap region of the retinal fundus image after registration which may be defined by a mask of a field of view of the retina after registration.

According to any one of the above aspects, detection of one or more pathology or indication of one or more pathology, disease or condition may include detection of one or more drusen and/or one or more bright lesion.

The detection of the one or more other bright lesions may include detection of one or more exudate and/or one or more cotton wool spot.

According to any one of the above aspects, a time between consecutive images in the two or more images may include one week; two weeks; three weeks; one month; two months; three months; four months; five months; six months; seven months; eight months; nine months; ten months; eleven months; one year; one and a half years; two years; two and a half years; three years; four years; five years or more than five years.

According to any one of the above aspects, a time between any two consecutive images in the two or more images may be different to the time between any other two consecutive images. The two or more images may be arranged in chronological order. Individual images within the two or more images may be referred to as 300i; 300ii; 300iii, etc.

According to any one of the above aspects, one or more parameter may be extracted and/or provided that may be used in a report. The report may quantify change in the one or more drusen and/or retina over the time period of the two or more images.

According to any one of the above aspects, the one or more drusen and/or one or one or more bright lesions may be tracked in a longitudinal study to observe disease or condition progression or a predisposition thereto.

According to any one of the above aspects, each of the two or more images may include a retinal image. Each retinal image may include a fundus image captured with a fundus camera. The fundus image may include a colour fundus image.

According to any one of the above aspects, each of the two or more images may be captured locally or received from one or more local or remote source. The one or more local or remote source may include a local or remote database or a local or remote image capture device such as, a local or remote fundus camera.

According to any one of the above aspects, if a respective one or more drusen and/or one or more bright lesions disappears it may not be detected in one or more of the two or more images. Additionally, one or more drusen and/or one or more bright lesions may be present in one or more later image but may be absent from one or more earlier images included in the two or more images.

According to any one of the above aspect, each, one or more, or respective drusen and/or bright lesions may be tracked. The tracking may include a timecourse.

According to any one of the above aspects, the providing may include obtaining one or more parameters of the area of newly appeared, disappeared and steady drusen and/or one or more bright lesion between two or more consecutive images in the two or more images. The one or more parameter may include one or more of size; shape; width; length; surface area; and volume.

According to any one of the above aspects, registration may include four modules. The four modules may include one or more of vessel segmentation; detecting and determining; matching branch points among the two or more images; and determining a best registration method. The detecting and determining may include detecting one or more branch points of the segmented vessels and determining one or more parameters of the detected one or more branch points. The matched branch points may include potential matched branch points. The determining may include determining a best registration method based on the matched branch points and deciding the success or rejection of the registration methods. Suitable registration methods include rigid; affine; projective; and quadratic methods.

According to any one of the above aspects, detecting and determining may include determining one or more parameter of the detected one or more branch points; removing one or more pixels from the one or more vessel center line which are not necessary to keep the center line contiguous; and defining one or more pixels which have more than two neighbor pixels as branch points. Determining may include determining an angle of one or more vessel center line. The angle may be determined with respect to the x-axis and width of the one or more vessel. The angle of the one or more center line may be determined by applying a line on one or more center line pixels. The applied line may include a first-order polynomial line. The applied line may be applied on one or more pixels of the center line. The applied line may be applied on 5; 10; 15; 20; 25; 30; 50 pixels. If another branch point is located within the pixels of the center line on which the applied line is applied, then the applied line may be utilized along a length until another branch point is reached for computing the angle of the center line. In other embodiments 5; 10; 15; 25; 30; 50 pixels may be used.

According to any one of the above aspects, the one or more parameter of the detected one or more branch points may include vessel width. The vessel width may be determined by iteratively removing the outer border of the one or more vessel to the center line. The iterative removing may include removing two pixels in each iteration. The two pixels removed may include top and bottom border pixels of the vessel. The number of removed pixels may be assigned as the width of the one or more vessel at each center line pixels. The median value of the one or more vessel segment may be used for the width of that respective vessel. The feature values may be saved after sorting according to angle.

According to any one of the above aspects, matching potential corresponding branch points within two images may include one reference image and a subsequent image transformed to be like the reference image. The one or more branch points may be matched.

According to any one of the above aspects, the matching branch points may include finding an alignment between the determined one or more parameters of two branch points, one in a reference image and the other in a subsequent image. The alignment may be found by minimizing the difference between the one or more parameters of two branch points. The difference may be minimized between the determined one or more angles. The matching may further include ensuring the order of the center line is same or unchanged.

The matching branch points may further include computing one or more metric values for each aligned pair of branch points. The one or more metric values may include four metric values. The four metric values may include: standard deviation of the difference of angles (σang); median of the difference of angles (Mang); standard deviation of the ratio of the width (σwid); and mean of the ratio of the width (Mwid) between the feature values of the branch points for the images.

The matching branch points may further include finding potential corresponding pairs of branch points from a reference image and a subsequent image included in the two or more images, wherein if two branch points of two images have σang<δang; and σwid<δwid; where δang and δwid are two threshold values for the difference of angle and the ratio of the width, they are selected as a potential corresponding branch points.

The matching branch points may further include finding one or more additional pairs of branch points between the two or more images. The finding one or more additional pairs may be continued iteratively until there is no additional or new pair satisfying the conditions.

The matching branch points may further include forming a list including potential corresponding branch points.

According to any one of the above aspects, determining the best registration method may include using the matched branch point list to find the best transformation matrix using different registration methods. If a list of potential matched branch points does not have enough points for any registration method then that registration method is skipped for that list and continue to the next list. Determining the best registration method may include determining a method which gives maximum overlap. The maximum overlap may be computed using Equation (1) where tp, fp and fn are true positive, false positive and false negative respectively, and c is the value for overlapping, higher c means higher overlap:

c=tp/(fp+fn) Equation (1).

According to any one of the above aspects, one or more non-drusen feature and/or non-bright lesion may be removed. The one or more non-drusen feature may include an Optic Disc.

According to any one of the above aspects the detection of one or more drusen and/or one or more bright lesion may include one or more of an intensity ratio between one or more of neighbor pixels; boundary edge; and/or OD.

According to any one of the above aspect, any change from two drusen containing segmented images in a common shared area may be quantified. The common shared area may be an overlapping region of the retinal fundus images after registration which may be defined by a mask of the field of view of the retina after registration.

The detection of the one or more drusen and/or bright lesion may include combining two or more detection methods. One detection method may be based on intensity ratio which detects one or more area with a brighter region compared to surroundings. Another detection method may be based on drusen boundary edge which detects one or more area with a sharp edge.

According to any one of the above aspects, intensity ratio detection may include a Gaussian filter to remove noise and a Gaussian filter and a mean filter to filter the filtered image. The intensity ratio detection may further include pixels having a ratio value more than one being detected as one or more drusen or one or more bright lesion.

According to any one of the above aspects, edge pixels in an image may be computed from the image gradient value and/or the standard deviation of the image. In a preferable embodiment, the edge pixels in an image are computed from the image gradient value of the image. The edge pixels in a potential drusen area or a potential bright lesion may be kept and a morphological filling operation may be applied to fill the close region by the edge pixels.

According to any one of the above aspects, the detected Optic Disc area and/or pixels located at the border of the image may be removed.

According to any one of the above embodiments, the one or more drusen and/or one or more bright lesions may be detected using one or more boundary edge pixels. The detection using one or more boundary edge pixel may include detecting one or more seed points, detecting one or more boundary pixels around the seed points and identifying regions that are brighter than background intensity as one or more drusen and or one or more bright lesion.

According to any one of the above aspects, the intensity ratio detection and the boundary based detection may be combined.

According to any one of the above aspects, one or more seed point may be detected by detecting a center of one or more drusen. The center may be detected by detecting a pixel which includes a local peak intensity in both x and y axis. A Gaussian filter with 5×5 window and 2 standard deviation may be applied for smoothing the image and reducing the intensity distortion effect of noise and any one or more pathology or any indication of one or more pathology, disease or condition present in the normalized image. A pixel may be defined as a local peak if it has higher or equal intensity than its neighbor pixels.

According to any one of the above aspects, the edge around one or more drusen and/or one or more bright lesions edge around each seed point may be detected and determined as a potential region of one or more drusen. The edge may be detected. The one or more edges may be detected with Canny edge detection algorithm.

According to any one of the above aspects, after detecting the one or more edges, all edge pixels around each seed point may be found. All edge pixels may be found by defining nearest edge pixels in 0 to 360-degree angle from each seed points as edge pixels around the pixel. All edge pixels may be found by using a shortest path algorithm

According to any one of the above aspects, any potential region which is not one or more drusen may be eliminated. The elimination may be by calculating a ratio between potential regions and the background. If a region includes a ratio greater than 1 it may be defined as one or more drusen otherwise it may be removed as a wrongly identified region. The value of background intensity may be defined by the mean intensity plus two standard deviations of the intensity of the image except for the potential regions. Then the pixel intensity of the potential regions may be divided by the background intensity. This output may then be combined with the intensity ratio based drusen detection to obtain the final output.

According to any one of the above aspects, the quantification may include a number of computations from two drusen-segmented images. The quantification may include using an overlap of a mask of the field of view of the two or more images and determining the shared area where the parameters of the changes in the one or more drusen and/or one or more bright lesions are quantified. In one embodiment (r−m)>0 may provide a disappeared area of drusen and (m−r)>0 may provide newly appeared area of drusen. A steady or unchanged area of drusen may be determined by an ‘AND’ operation (m&r).

The one or more pathology or an indication of one or more pathology, disease or condition progression, may include an ocular pathology or indication of one or more ocular pathology, disease or condition progression. The ocular disease or condition may include any ocular disease or condition and may for example include one or more of age-related macular degeneration (AMD); glaucoma; angioid streaks; retinitis pigmentosa; glaucoma and/or neoplasm of the choroid, cranial nerves, retinal or eyeball.

Further, any prior art reference or statement provided in the specification is not to be taken as an admission that such art constitutes, or is to be understood as constituting, part of the common general knowledge.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the disclosed technology will be obtained by reference to the following detailed description that sets forth illustrative embodiments, in which the principles of the technology are utilized, and the accompanying drawings.

FIGS. 1A and 1B: Drusen changes shown in red-free fundus image between two visits. The left image, FIG. 1A, shows image 300i captured during a first visit, and the right image, FIG. 1B, shows image 300r, captured during a second or other follow up visit after registration. The top arrow (red) shows disappearance of drusen, the encircled region (inside red circle) shows newly appeared drusen, the right (yellow) arrow shows splitting of drusen, and middle (blue) arrow shows one or more drusen with a temporal intensity difference.

FIG. 2: This shows a flow diagram showing one embodiment of method according to the disclosure.

FIGS. 3A and 3B: The retinal color fundus image (left; FIG. 3A) and vessel segmentation using U-net (right; FIG. 3B).

FIGS. 4A and 4B: The segmented vessels of a cropped retinal image (left) and the center line of the vessel (right), are shown. The (blue) star in both images is the branch point. The fitted line in the top right segment (green), the fitted line in the top left segment (red) and the fitted line in the bottom left segment (blue) are the first order polynomial line of the corresponding center lines. The determined one or more parameters of this branch point are=(angle1; angle2; and angle3; and width1; width2; and width3). For this example, the determined one or more parameters are (164.44; 37.69; and 261.03; and 6; 13; and 12).

FIGS. 5A and 5B: These images show the determined one or more parameters for the same branch point in two images. For example, the left image is from a reference image 300i, and the right image is from a subsequent image 300ii. The corresponding center line is marked with the same color in respective images. The determined one or more parameters of the branch point of the reference image 300i are (164.44; 37.69; and 261.03; and 6; 13; and 12) and the subsequent image 300ii is (161.57; 32.01; and 249.15; and 8; 12; and 12).

FIG. 6: The registration output of two colour fundus image using a checkerboard.

FIGS. 7A and 7B: These images show locations where a user has manually clicked on corresponding pixels in two images 300i (FIG. 7A, left) and 300ii (FIG. 7B, right).

FIGS. 8A-8C: These show cropped images. FIG. 8A shows points at which a user has manually clicked pixels (left image). FIG. 8B shows an image on which vessel segmentation has been performed and the clicked pixels maintained (middle image). FIG. 8C shows the same image on which a vessel center line has been calculated and superimposed (right image). Bottom (blue) star and top (red) star are manually clicked points and branch points selected as a pair on image 300i.

FIGS. 9A and 9B: These show images which visualize the output of automatic registration (left; FIG. 9A) and semi-automatic registration (right; FIG. 9B).

FIGS. 10A-10D: The receiver operating characteristic (ROC) curves for the state-of-the-art methods including the proposed method on differently characterized images within a data cohort.

FIG. 11: Flow diagram of tracking the changes of drusen using retinal fundus image from multiple visits according to one embodiment of the disclosure.

FIGS. 12A-12D: A) The potential drusen area, B) edge pixels in the potential drusen area, C) filled the image of B), and D) the drusen after removing the optic disc.

FIG. 13: The potential seed points of the drusen.

FIGS. 14A and 14B: A) shows Edge pixels (white); Seed point (green); 0, 45 and 90 degree angle lines (blue) from seed point; and nearest edge pixels (red) along those lines. B) shows longest line (red) of nearest edge pixels which cover 90 degree angle distance.

FIGS. 15A and 15B: A) shows Edge pixels (red) using Canny Edge Detection Algorithm. B) nearest edge pixels (red) from a seed point (green).

FIGS. 16A and 16B: A) shows the potential region (red) polygon. B) shows all potential regions in the image.

FIG. 17: An fundus image showing an example of the one or more drusen detected with the proposed method.

FIG. 18: This shows one embodiment of a computer system suitable for the disclosure.

FIG. 19: This shows a schematic block diagram of one embodiment of a processor suitable for use in the present disclosure.

FIG. 20: This shows a schematic diagram of one embodiment of the disclosure utilizing a telemedicine or remote medicine platform.

FIGS. 21A-21L: These show screen shots of one embodiment of a graphical user interface (GUI) for using the method and system of the disclosure.

Skilled addressees will appreciate that elements in the drawings are illustrated for simplicity and clarity and have not necessarily been drawn to scale. For example, the relative dimensions of some elements in the drawings may be distorted to help improve understanding of embodiments of the present disclosure.

### DETAILED DESCRIPTION

The following description refers to specific embodiments of the present disclosure and is in no way intended to limit the scope of the present disclosure to those specific embodiments.

For the purpose of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Various alterations, rearrangements, substitutions, and modifications of the features illustrated herein, and any additional applications of the principles of the present disclosure as illustrated herein, which would occur to one skilled in the relevant art and having possession of this disclosure, are to be considered within the scope of the present disclosure.

As used herein “image registration” is used to mean transforming two or more sets of image data into one coordinate system. An example of a set of image data is a digital image such as, a fundus image.

As used herein “timecourse” is used to mean the variation of a quantity or quality over time. This is understood to be a meaning used in science as opposed to a conventional medical use, or meaning, which refers to the varying activity of a medicine over time following administration. According to the meaning adopted herein, the first one elucidated in this paragraph, in one embodiment the quantity that varies over time is one or more drusen.

As used herein “drusen” is used to mean accumulations of extracellular material that build up in the eye. The term “drusen” is plural and the singular is the term “druse”. The druse or drusen may be yellow or white in colour. The druse or drusen may accumulate between the Bruch's membrane and the retinal pigment epithelium (RPE) of the eye. It is generally accepted that the presence of larger and more numerous drusen in the macula is a common early sign of age-related macular degeneration (AMD).

As used herein the term “pathology” is used to mean any deviation from a healthy or normal condition. In one embodiment, a pathology may be indicated or associated with one or more bright lesion of the retina.

The term “lesion”, as used herein, means any localized, defined area of diseased tissue or tissue with any deviation from a healthy or normal condition.

In one embodiment, the present disclosure provides image registration for finding one or more pathology or an indication of one or more pathology, disease or condition progression analysis. The indication of one or more pathology, disease or condition may include one or more drusen and/or one or more bright lesion in the retina. A novel registration technique utilizing one or more of: U-net based retinal blood vessel segmentation; vessel centerline computation; detecting and extracting parameters of the branch points of the vessels; finding potential matched branch point lists among the two images; and determining the best registration method such as, rigid; affine; projective; and quadratic, based from the matched branch points and deciding the success or rejection of the methods is provided.

The one or more parameters may include one or more of size; shape; width; length; surface area; and volume.

A semi-automatic retinal color fundus image registration method is also provided. The registration method may include a user directed selection. The user directed selection may include a point or region of interest which may include one or more pixels which correspond to a branch point. The one or more pixels may be selected by a user who clicks or otherwise selects the one or more pixels as a point or region of interest. The user selection may be utilized when the automated system (e.g. using the U-net algorithm) fails, which may occur, for example, due to noise or a poor quality image. First, a user may click on or otherwise select the same point or region in two images of a pair of images. Then a small region, for example, 100×100 pixels, may be cropped at the click or otherwise selected point and vessel segmentation may be determined using an intensity ratio between an average filtered image with large window, for example, 20×20, and Gaussian filter image with large standard deviation. Then the nearest branch point may be determined from the reference click or otherwise selected point or region and may be stored in a list named MP (a variable for a matched point). The best registration method may be determined and a transformation matrix applied as described with respect to the automatic method.

Although one embodiment of the disclosure will be described with reference to one or more drusen, the disclosure is not so limited. In other embodiments, one or more bright lesions in the retina.

Also provided is a drusen segmentation and quantification method. This method may be based on one or more of intensity profiling; boundary edge detection; shape analysis; vessel segmentation; optic disc detection; and optic disc removal. Two approaches may be used to detect the drusen and they may be combined into a final output. The first approach may include an intensity ratio between surrounding pixels. The second approach may include finding regions based on boundary edge pixels and the intensity ratio between neighbor pixels and the potential drusen area around the seed point, then merging these regions, defining all of them as a drusen area, and quantifying the total number of pixels. This may include performing shape analysis using an intensity ratio between neighbor pixels and the potential drusen seed points. The drusen area may be segmented by merging the intensity and edge-based region identification and the total number of pixels may be quantified.

A method of one or more pathology or an indication of one or more pathology, disease or condition progression analysis is also provided. In this method the same pathological feature or indication of pathology, disease or condition is found on two or more images through a seed point of the corresponding pathological feature or indication of pathology, disease or condition. Overlapping regions within the registered images and the area changes may be determined by computing the change in an area of pathology or indication or one or more pathology, disease or condition from a first visit to a second visit. The parameters of the area of newly appeared, disappeared and steady pathological feature or indication or pathology, disease or condition between the images may be extracted. After registration and detection, the changes from two segmented images may be determined in the common shared area. The common shared area may be the overlap region of the retinal fundus image after registration which is defined by the mask of the field of view of the retina after registration.

It is desirable to track the change in any pathology or indication of pathology, disease or condition observable. This is particularly true in ocular disease where loss of sight is of particular concern. One example of ocular disease is retinal disease which may be tracked through changes in the retina. In one embodiment of the disclosure, one or more retinal color fundus image is used to track an observable pathology or condition or indicator of pathology or condition. The indicator of pathology or condition may include drusen.

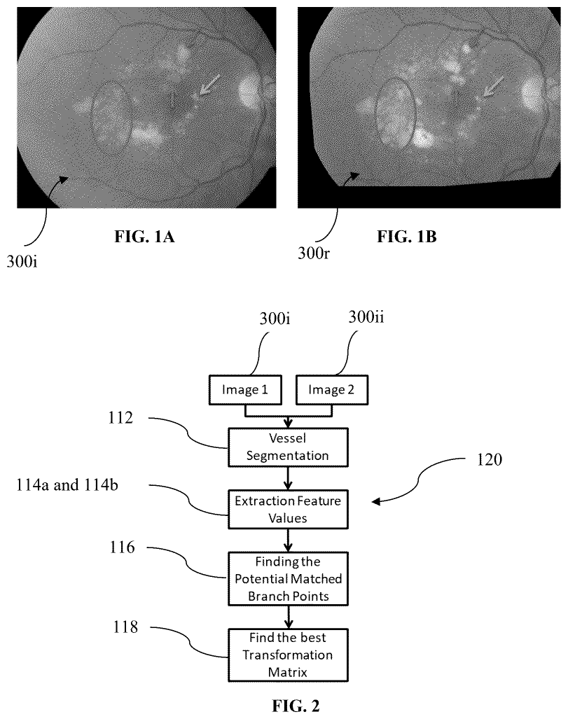

Suitable retinal colour fundus images 300i, 300r are shown in FIGS. 1A and 1B. The follow up image, which is registered to the previous imaged 300i, was captured one year (12 months) after the capture of FIG. 1A which shows image 300i. That is, FIG. 1 shows image 300i and FIG. 1B shows image 300r which shows image 300i registered to image 300ii. An example of one or more indicator of pathology or condition that may be tracked is the change in one or more drusen in images taken in follow up visits. The observation of drusen is regarded as being important in the identification of progression to late age-related macular degeneration (AMD). Two or more images 300 may be taken at discrete times, that is during different visits for image capture and analyzed to detect any progression.

The time between consecutive images in the two or more images 300 may be one week; two weeks; three weeks; one month; two months; three months; four months; five months; six months; seven months; eight months; nine months; ten months; eleven months; one year; one and a half years; two years; two and a half years; three years; four years; five years or more than five years.

The time between any two consecutive images in the two or more images 300 may be different to the time between any other two consecutive images. The two or more images 300 may be arranged in chronological order. Individual images within the two or more images 300 may be referred to as 300i; 300ii; 300iii, etc. 300r is used to refer to one or more image that includes two or more data sets that have been transformed into one coordinate system in image registration.

The phrase “a plurality of the two or more images” is used herein to mean any two or more images within the set of two or more images 300. The plurality of two or more images may be a subset of the two or more images 300 or may include the complete set of the two or more images 300. The plurality of the two or more images may include any two or more images within the set, whether consecutive or not.

There are many challenges to finding the same area in two or more images taken at different times. These challenges include: image variability caused by the operating environment; the image capture angle; the image capture distance; patient position; operator attention; and camera used. These challenges must be overcome if the two or more images taken at different times are to be transformed into one coordinate system in the process of image registration.

Once image registration has been accomplished, another challenge is automatic pathology or indicator of pathology, disease or condition quantification. In one embodiment, the automatic pathology quantification may include detection of one or more drusen or any other bright or white pathology or indication or pathology such as, one or more exudate and/or one or more cotton wool spot. In this embodiment, challenges may include: one or more of different size of one or more drusen; different color of one or more drusen; different intensity of one or more drusen; different contrast of one or more drusen; one or more artefact; and noise in one or more image.

An advantage of the present disclosure is that one or more parameter is extracted and/or provided that may be used in a report. The report may quantify change in the one or more drusen and/or retina over the time period of the two or more images 300.

In one embodiment, the present disclosure provides a method 100 for quantifying change in one or more drusen over a time period. Advantageously, method 100 may allow tracking of the one or more drusen in a longitudinal study to observe disease or condition progression or a predisposition thereto.

Method 100 includes registering 110 two or more images 300 wherein each image is taken at a discrete time.

Each of the two or more images 300 may include a retinal image. Each retinal image may include a fundus image captured with a fundus camera. The fundus image may include a colour fundus image. The registration 110 may include identifying a same or identical reference location of a retina in each of the two or more by images 300. Each of the two or more images 300 may be captured locally or received from one or more local or remote source. The one or more local or remote source may include a local or remote database or a local or remote image capture device such as, a local or remote fundus camera.

Method 100 also includes detecting 120 the one or more drusen in one or more of the two or more images 300. If one or more drusen disappears it may not be detected in one or more of the two or more images 300. Additionally, one or more drusen may be present in one or more later image 300 but may be absent from earlier images 300.

An additional step in method 100 is locating 130 the detected one or more drusen in each of the two or more images 300. The locating 130 may further include tracking the location of each respective one or more drusen

The ability to determine the location of the one or more drusen in the two or more images 300 is of significant advantage because it then allows each, one or more, or respective drusen of the one or more drusen to be tracked. The tracking may include a timecourse.

Another step in method 100 is quantifying 140 the located one or more drusen in each of the two or more images 300.

Method 100 also includes providing 150 one or more parameters of the quantified one or more drusen to thereby quantify change in one or more drusen over the time course of the two or more images 300.

The providing 150 may include obtaining one or more parameters of the area of newly appeared, disappeared and steady drusen between two or more consecutive images in the two or more images 300.

### Image Registration Methodology

Image registration is an important computer vision problem because it gives the ability to extract more information from a longitudinal study as well as overcomes hardware limitations by creating mosaics to allow the production of a large image, such as, for example, a full structure of any anatomical feature such as, the retina.

In one embodiment, the disclosure provides registration 110 of two or more retinal fundus images.

In another embodiment, registration 110 includes four main modules. One module includes vessel segmentation 112. Another module includes detecting and determining 114. The detecting and determining 114 includes detecting one or more branch points of the segmented vessels and determining one or more parameters of the detected one or more branch points. Yet another module includes matching 116 branch points among the two or more images 300. The matched branch points may include potential matched branch points. Still another module includes determining 118 the best registration method based on the matched branch points and deciding the success or rejection of the registration methods. Suitable registration methods include rigid; affine; projective; and quadratic methods.

FIG. 2 shows a flow diagram depicting one embodiment of registration 120 according to method 100. Image 1, 300i, and Image 2, 300ii, are two individual images within the two or more images 300.

Below further modules included in method 100 are described.

### Vessel Segmentation

There are many algorithms available for retinal vessel segmentation from fundus images for example those provided in References [1] to [3]. In one embodiment, method 100 includes U-Net, a deep learning algorithm for biomedical image segmentation, for retinal vessel segmentation, see Reference [2].

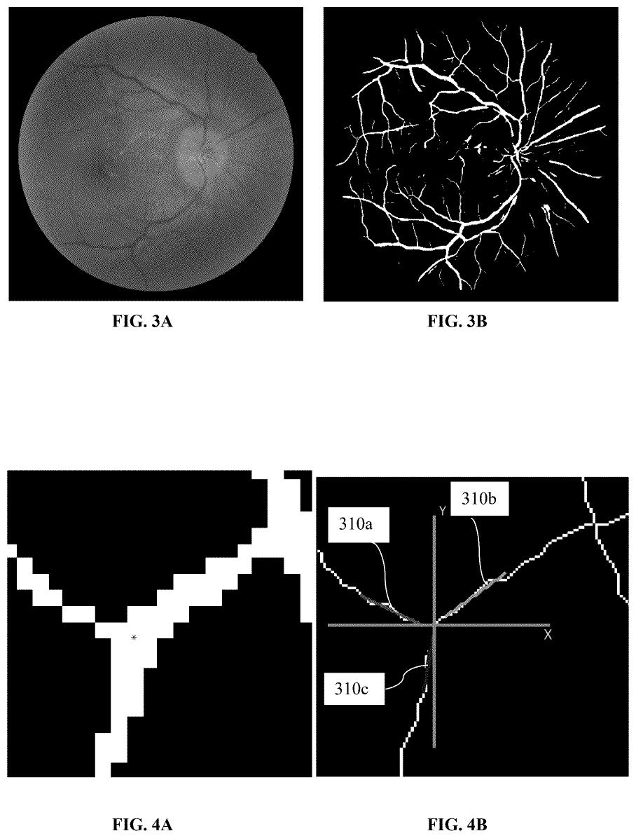

U-Net is a fully convolutional network in which the training strategy relies on the strong use of data augmentation. Hence, high accuracy can be achieved using a small number of training images. The network may include a contracting path to capture context and a symmetric, expansive path which enables precise localization. The contracting path may include two 3×3 convolutions (unpadded convolutions), each followed by a rectified linear unit (ReLU) and a 2×2 max pooling operation with stride 2 for down-sampling. In every step of the expansive path, pooling operators may be replaced by up-sampling operators, which allows the network to propagate context information to higher resolution layers. In the final layer, a 1×1 convolution is used to map each component feature vector to the desired number of classes. In total the network may include 23 convolutional layers. This algorithm has been tested on the DRIVE; STARE; CHASE_DB; and HRF dataset and was determined as the best method, similarly by Reference [4]. FIGS. 3A and 3B show an example of an output using the U-net.

### Detection of One or More Branch Points and Determining One or More Parameter of the Detected One or More Branch Points

After segmenting 112 a vessel, a robust algorithm may be developed and/or applied for detecting and determining 114. Detecting and determining 114 may include determining 114a one or more parameter of the detected one or more branch points; removing 114b one or more pixels from the one or more vessel center line which are not necessary to keep the center line contiguous; and defining 114c one or more pixels which have more than two neighbor pixels as branch points.

In detecting and determining 114 some of the crossover points may be defined as branch points. This is not problematic because an aim is to find the potential corresponding points in the two images, for example 300i, 300ii, within the two or more images 300.

Determining 114a may include determining an angle of one or more vessel center line with respect to the x-axis and width of the one or more vessel. The angle of the one or more center line may be determined by applying a first-order polynomial line 310 on 20 pixels of the one or more center line pixels. FIGS. 4A and 4B show one example of determining 114a wherein the fitted line in the top left segment (red) in FIG. 4B, 310a; the fitted line in top right segment (green), 310b, in FIG. 4B; and the fitted line in bottom left segment (blue), 310c, in FIG. 4B; are first order polynomial lines on the corresponding center lines and angles are 164.44; 37.69; and 261.03, respectively. If another branch point is located within these 20 pixels, then the fitting line may be utilized along a length until another branch point is reached for computing the angle of the center line. In other embodiments 5; 10; 15; 25; 30; 50 pixels may be used.

The one or more parameter of the detected one or more branch points may also include vessel width. In this respect, the inventors have also developed a robust algorithm for determining the width of the one or more vessel. First, the outer border of the one or more vessel is iteratively removed to the center line, as a result two pixels, top and bottom border of the vessel, are removed in one iteration. The number of removed pixels is assigned as the width of the one or more vessel at each center line pixels. The median value of the one or more vessel segment, i.e., the pixels used for computing the angle, is used for the width of that respective vessel. So, if a branch point has three center lines, then it includes six features encompassing of three angles and three vessel widths, as shown in FIGS. 4A and 4B. Similarly, if a branch point has four vessel center lines, then it will have four angles and four vessel widths. Finally, the feature values may be saved after sorting according to angle.

### Matching Potential Corresponding Branch Points

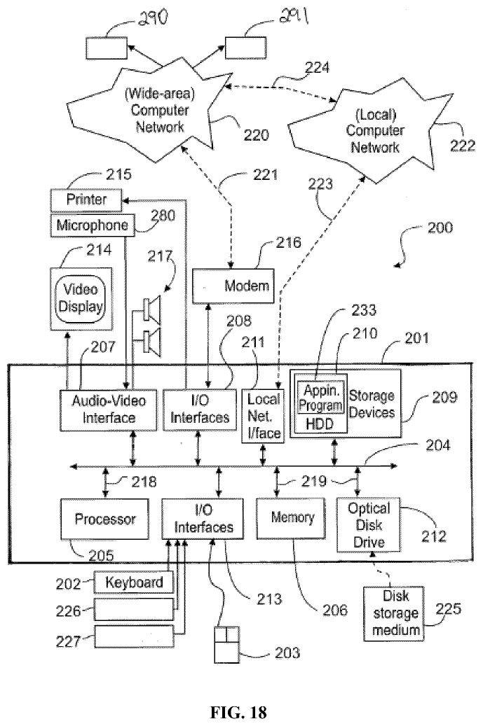

After detecting and determining 114 one or more parameters of the detected branch points, method 100 matches 116 potential corresponding branch points within two images 300i, 300ii; one is a reference image 300i, and another one is a subsequent image 300ii which has been transformed to be like the reference image 300i. The inventors have developed a new and robust algorithm for finding these points, and the pseudo code is provided in Algorithm 1, see FIG. 18.

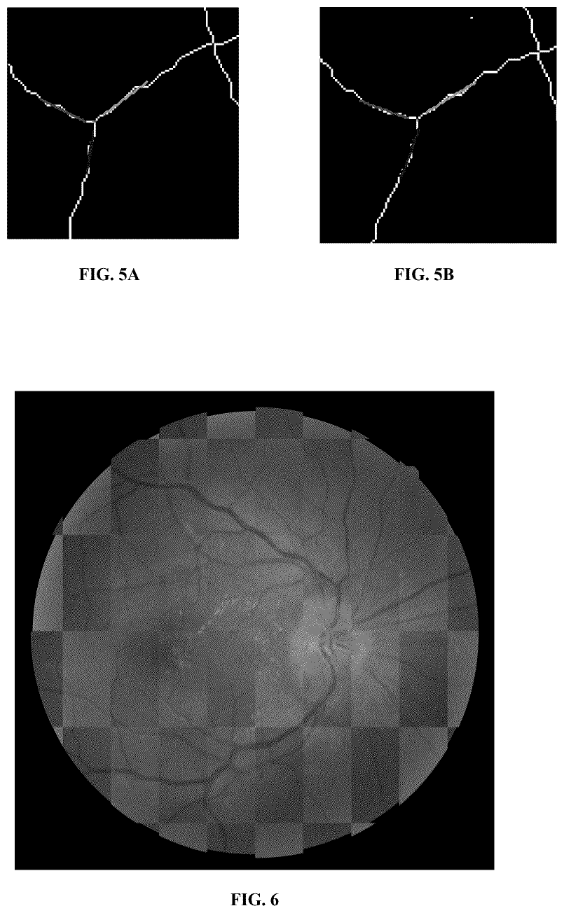

The first step is finding an alignment between the determined one or more parameters of two branch points, one in reference image 300i and the other in subsequent image 300ii. In one example, the one or more parameters of a branch point in reference image 300i are (164.44; 37.69; and 261.03; and 6; 13; and 12;), see FIG. 5A, and the one or more parameters of a branch point in subsequent image 300ii are (161.57; 32.01; and 249.15; and 8; 12; and 12], see FIG. 5B. The alignment may be found by minimizing the difference between the one or more parameters of two branch points. In one embodiment, the difference is minimized between the determined one or more angles. At the same time, it may be ensured the order of the center line is same. For example, with respect to FIGS. 5A and 5B, the alignment order is [1, 2, 3] for the reference image 300i and [1, 2, 3] for the subsequent image 300ii.

If the image is rotated 50 degrees more, then the one or more parameters of the subsequent image 300ii become [101.57; 199.15; and 341.99; 8; 12; and 12] where the first centerline becomes the third; the second becomes the first; and the third becomes the second due to sorting according to angles. In this case, the best alignment is found [1, 2, 3] for the reference image 300i and [3, 1, 2] for the subsequent image 300ii. Here, the order of the vessel may also be maintained.

The second step is computing one or more metric values for each aligned pair of branch points. In one embodiment, the one or more metric values includes four metric values including: 1) the standard deviation of the difference of angles (σang); 2) the median of the difference of angles (Mang); 3) the standard deviation of the ratio of the width (σwid); and 4) the mean of the ratio of the width (Mwid) between the feature values of the branch points for the images.

The third step is finding potential corresponding pairs of branch points from the reference image 300i and subsequent image 300ii. If two branch points of two images have σang<δang; and σwid<δwid; where δang and δwid are two threshold values for the difference of angle and the ratio of the width, they are selected as a potential corresponding branch points and a new list is formed.

Next another pair of branch points between image 300i and 300ii which satisfy the above conditions as well as maintaining conditions satisfied in the third step, wherein a pair was selected as a potential matched pair, is found, see Algorithm 1 (FIG. 18). This process is continued iteratively until there is no additional or new pair satisfying the conditions and separately a list is formed. This way a list including potential corresponding branch points is obtained which may be used to find the best transformation matrix.

### Determining the Best Registration Method

In determining step 118, the matched branch point lists are used to find the best transformation matrix using different registration methods. Examples of registration methods include: rigid; affine; projective; and quadratic registration methods. If a list of potential matched branch points does not have enough points for any registration method such as, rigid requires 2 points; affine requires 3 points; projective requires 3 points; and quadratic requires 4 points, then that registration method is skipped for that list and continue to the next list. In one embodiment a method is used which gives maximum overlap. The best overlap may be computed using Equation (1) where tp, fp and fn are true positive, false positive and false negative respectively, and c is the value for overlapping, higher c means higher overlap. FIG. 6 shows an example of registration output using this method.

c=tp/(fp+fn) Equation (1)

### Semi-Automatic Registration Method

Though the automatic registration 110 described above has a high accuracy, sometimes it fails due to poor quality of the image. As a result, vessel segmentation is not good enough to detect the branch points. In this situation, a semi-automatic registration technique may be utilized. The procedure of the semi-automatic method may include finding corresponding branch points with a user click or other selection.

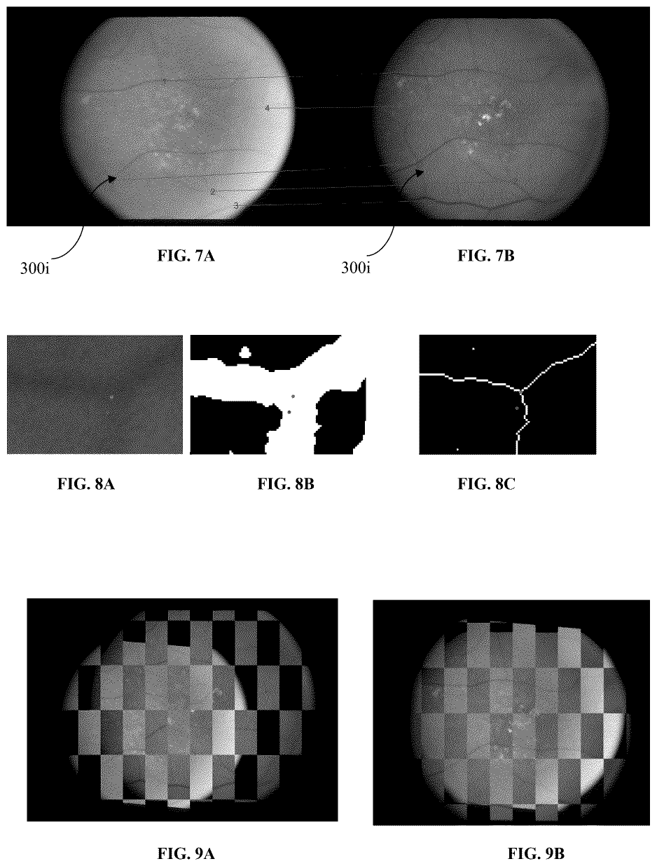

First, a user may click on or otherwise select at the same point or region within each of two images 300i, 300ii in the pair 300i,ii as shown in FIGS. 7A and 7B. Since, U-net does not perform well in these images 300i; 300ii due to poor image quality, in the second step, a small region (100×100 pixels) is cropped at the click point and the vessel segmentation is determined using an intensity ratio between average filtered image with a large window (20×20) and Gaussian filter image with large standard deviation (Equation 3). Then the nearest branch point is found from the reference click or otherwise selected point or region as shown in FIG. 8A; 8B; and 8C and stored in a list named MP, a variable for a matched point in Algorithm 1.

Lastly, the best registration method and transformation matrix may be determined as described in the automatic method. FIGS. 9A and 9B show an example of failed automatic registration and successful semi-automatic registration, respectively for the same image pair.

### Experimental Results on Image Registration

The automated method of image registration has been evaluated on a public dataset named FIRE which has available ground truth [5]. The dataset contains 134 pairs of retinal color fundus images 300i, 300ii, with 2912×2912 pixels each. The pairs of images 300i, 300ii are divided into three categories: S which includes 71 pairs of images 300i, 300ii with more than 75% overlap; P which includes 49 pairs of images 300i, 300ii with less than 75% overlap; and A which includes 14 pairs of images 300i, 300ii with more than 75% overlap and the presence of one or more pathology or indication of one or more pathology, disease or condition.

The inventors have followed the same approach for analyzing the accuracy of the proposed registration method 110 as followed in the FIRE dataset. The approach includes computing the mean distance from the manually selected one or more corresponding point or region. Comparison has also been performed with other state-of-the-art methods including the owner of the dataset; their results are available with the dataset described in Reference [5].

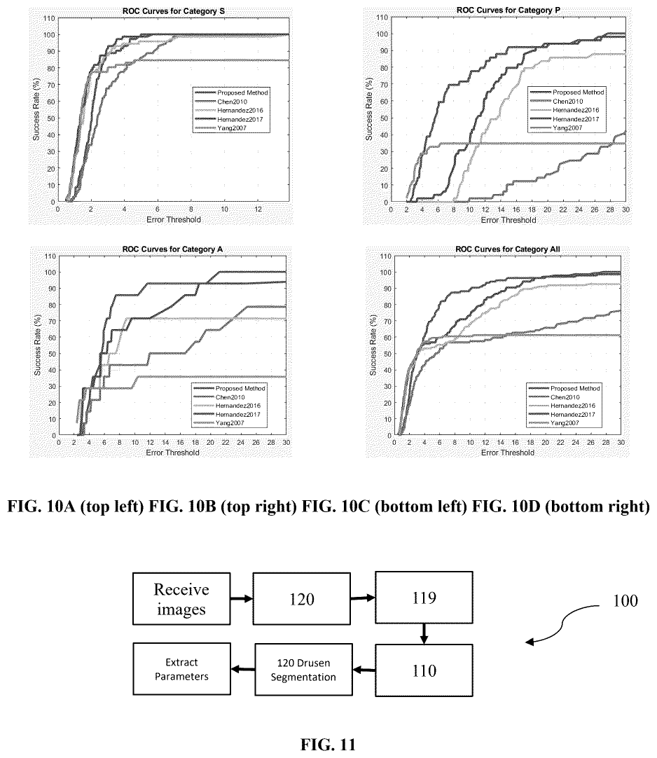

FIG. 10A; 10B; 10C; and 10D show the ROC curve of all methods including our method 100 (labelled “proposed method”) for each category as well as all images. This ROC curve indicates Hernandez and our proposed method 100 perform well in category S. All other cases, method 100 outperforms the other methods. In all images, method 100 achieved around 70% success rate with 4 pixels error while best state-of-the-art method achieves less than 60% success rate.

### Methodology for Detecting the One or More Drusen

Method 100 provided by the inventors allows the determination of one or more parameters to track the changes of drusen in retinal fundus images 300 in different visits. In one embodiment three parameters are determined. FIG. 11 shows a flow diagram illustrating steps in one embodiment of method 100.

First two or more images 300i, 300ii are obtained and/or received as described above. In step 120 vessel segmentation is performed also as described above. Since the optic disc (OD) is white, it can be mistaken as drusen. Therefore, in one embodiment, method 100 includes removing 119 one or more non-drusen feature such as, the OD. The removal may be by detecting the OD using the method described in Bhuiyan et al. [6] and the OD may then be removed from the region of interest (ROI) of drusen detection. Then the above registration step 110 is applied to obtain a common reference area within which one or more parameters of one or more drusen may be quantified.

In step 120 the one or more drusen are detected. The detection may use one or more of an intensity ratio between one or more of neighbor pixels; boundary edge; and/or OD.

After registration 110 and detection 120 of the one or more drusen, any changes from two drusen containing segmented images 300i, 300ii in the common shared area may be quantified. The common shared area is the overlapping region of the retinal fundus images 300i,ii after registration which is defined by the mask of the field of view of the retina after registration.

### Optic Disc Detection

Since drusen and optic disc are bright, it is desirable to isolate the OD when detecting 120 the one or more drusen. The inventors have adapted a method for OD detection which is based on image global intensity levels; OD size; and shape analysis [6]. The reasons for considering these features are as follows. Firstly, the OD is the brightest part of the image, and its pixel intensity values may be approximated by analyzing an image histogram. Secondly, the OD is more or less circular shaped, and its size may be specified within a particular range for any person. Therefore, incorporating size and shape information along with the pixel intensity would provide the highest accuracy in OD detection. FIGS. 3A and 3B show an example of detecting the optic disc using the combined method.

### Detecting the One or More Drusen

The inventors have proposed a simple, efficient and effective algorithm for detecting the one or more drusen. Drusen are typically brighter than their surroundings in the retinal fundus image 300. Another feature common or typical to drusen is that they have a sharp edge, otherwise that brighter region putatively identified as one or more drusen is either noise or pseudo-drusen, which we do not consider herein.

Two methods may be used to detect 120 the one or more drusen. These two methods may be combined to get the detected drusen area. The first method is based on intensity ratio which detects one or more area with a brighter region compared to surroundings and the second method is based on drusen boundary edge which detects one or more area with a sharp edge. These two methods are described below.

### Intensity Ratio Based Drusen Detection

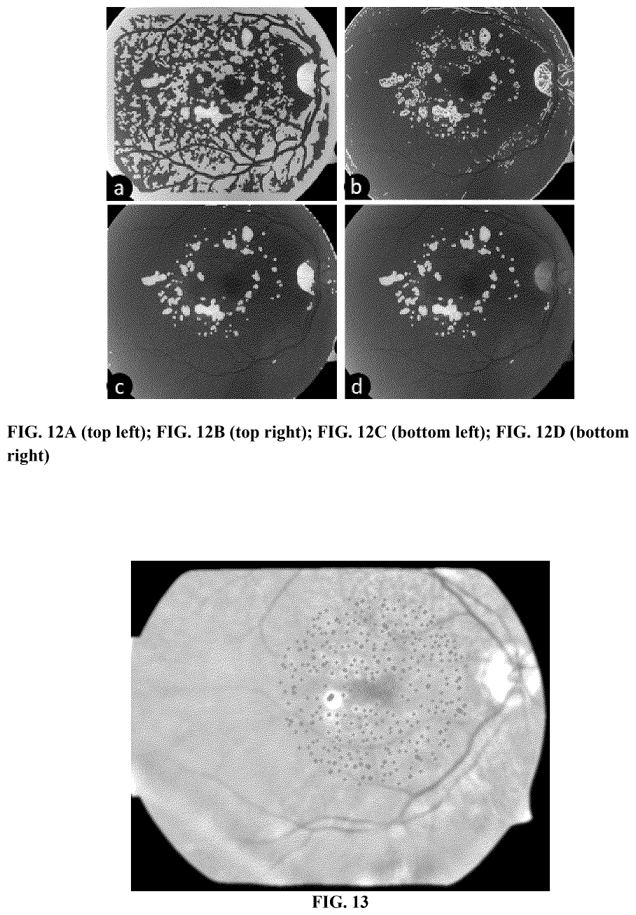

First, a Gaussian filter using σ=2 is used to remove noise, this may be referred to as “In”. Then a Gaussian filter with σ=5 and mean filter with 50×50 window are applied to that image 300. This operation blurs the entire image 300 using the surrounding pixel intensity, this may be referred to as “Is”. As a result, the brighter intensity of the original image 300 becomes a little bit darker, and darker intensity becomes a little bit brighter in the “Is”. So, in the ratio of “In” to “Is”, the pixel value for pixels of one or more drusen must be greater than 1. At the same time, some background pixels have a value more than one due to noise, artefacts and vessels, (see FIG. 12A). The potential drusen area may be approximated as the pixels having a ratio value more than 1.

Edge pixels in the image 300 may be computed from the image gradient value and/or the standard deviation. Preferably, edge pixels are computed from the image gradient value of the image. The edge pixels in the potential drusen area are kept (see FIG. 12B) and a morphological filling operation is applied to fill the close region by the edge pixels (see FIG. 12C).

The OD area that was detected in step 119 may be removed and the pixels located at the border of the image are also removed as shown in FIG. 12D. The remaining area may be defined as the one or more drusen.

### Boundary-Based Drusen Detection

The intensity ratio based drusen detection may sometimes miss some drusen which may be detected using boundary edge pixels. In this method, seed points may be detected, then the boundary pixels around the seed points may be detected and finally regions which are brighter than background intensity are identified as one or more drusen. Then this output may be combined with the intensity ratio based drusen detection to get the full one or more drusen segmentation.

Detection of the potential seed points: Typically, drusen have relatively higher intensity values and the center of each one or more drusen ordinarily includes the upper most intensity in its region. So the center of each one or more drusen is a pixel which includes a local peak intensity in both x and y axis. The potential centers of each one or more drusen may be detected as potential seed points. A Gaussian filter with 5×5 window and 2 standard deviation is applied three times for smoothing the image and reducing the intensity distortion effect of noise and any one or more pathology or any indication of one or more pathology, disease or condition present in the normalized image.

This smoothing operation may blur the edge of objects that are not suitable for border detection. Since an aim is finding the centers of one or more drusen, this blurring may help avoid finding incorrect centers of one or more drusen. This operation cannot avoid all incorrect centers due to the presence of noise and pathologies. The incorrect centers may be removed in the next steps.

In this step, a pixel may be defined as center of one or more drusen or a seed point if it is a peak in both x and y axis direction as shown in FIG. 13. The (green) dots on the image each represent respective drusen in the detected one or more drusen.

A pixel may be defined as a local peak if it has higher or equal intensity than its neighbor pixels.

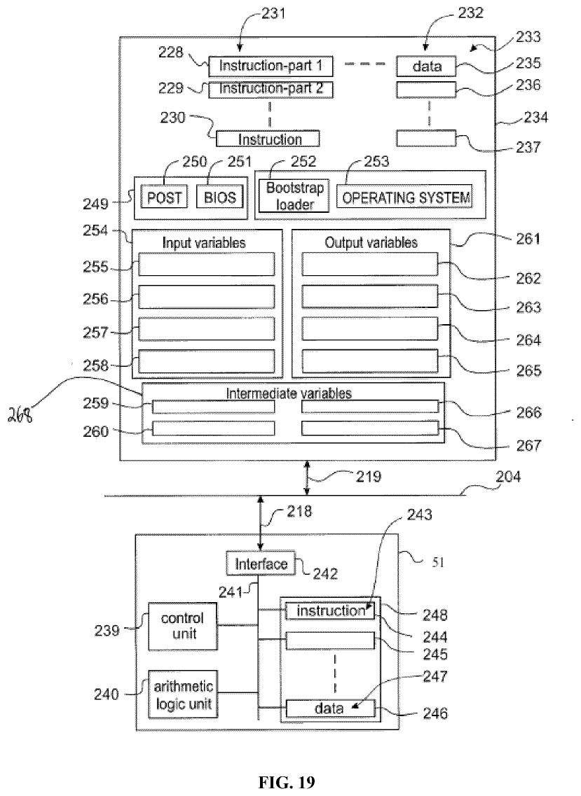

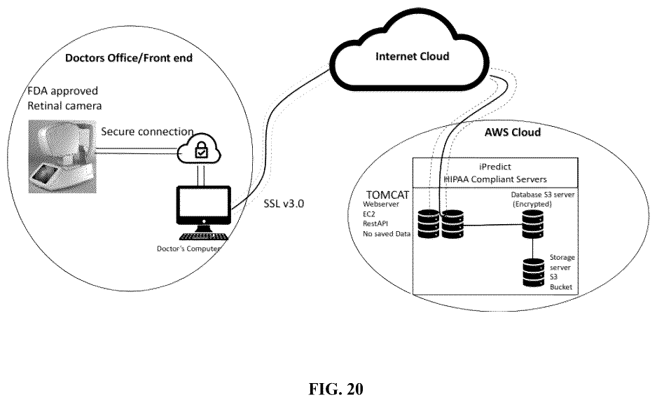

Potential Regions Finding around seed points as drusen: Druse are typically imaged as white pixels with an edge, the border of the druse, around it. The edge around each seed point may be detected which may be determined as a potential region of one or more drusen. The pseudo code for potential region finding around seed points as drusen is referred to as Algorithm 2 and is shown in FIG. 19.

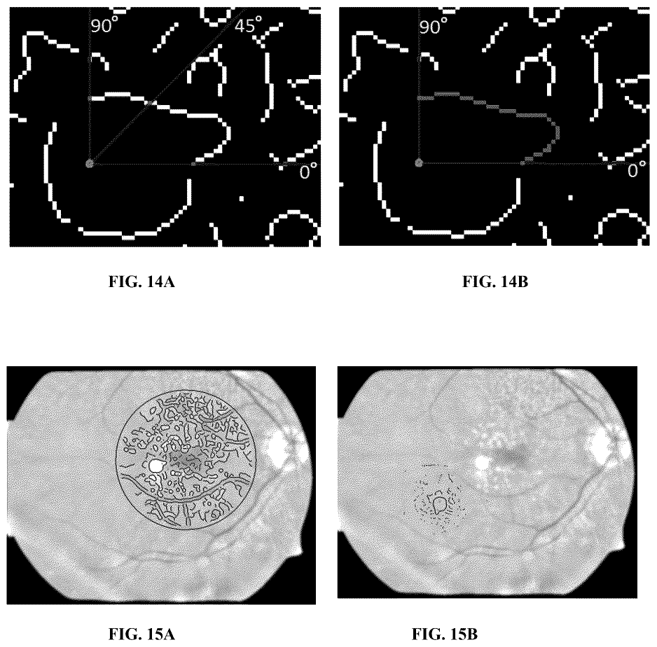

The Canny edge detection algorithm may be used to find one or more edges in an image 300 (see FIG. 15A). After detecting the one or more edges, all edge pixels around each seed point may be found. The nearest edge pixels in 0 to 360-degree angle from each seed points (see FIG. 14A) may be defined as edge pixels around the pixel as shown in FIG. 15B. This operation finds corresponding border pixels of the seed point as well as some unwanted pixels. To keep only border pixels of the seed point, a shortest path algorithm may be applied wherein each pixels are the graph nodes. There are two challenges to apply shortest path algorithm, and they are: 1) which are the start and end nodes for the graph; and 2) how can we prevent non-border pixels from the shortest path.

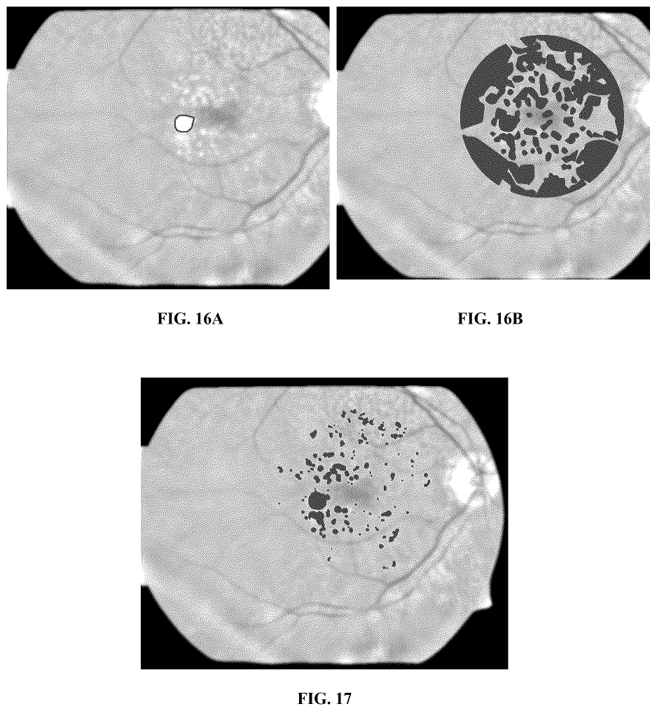

For the second challenge, we analyze the connected component of the graph. If two pixels Euclidean distance is less than 2 then they are connected, and connected pixels form the connected component. If there is not a single longest line (connected component) which covers at least 90-degree angle distance (see FIG. 14B) for the seed point, then it will be removed from the seed points. Otherwise, the end pixels of the longest line may be defined as start and end nodes of the graph. The pixels associated with the longest line may be removed from the graph so that shortest path detects a different path for the start and end nodes which will be another side of border edge pixels of the one or more drusen. The edge weight is the Euclidian distance from each pixel. Then the Dijkstra shortest path may be applied to find the shortest path. If there is no shortest path, then the seed point may be removed. Otherwise, a mathematical method such as, convex hull, may be applied on those edge pixels found with the shortest path and the connected component. At this stage, the regions from all seed points may be defined as a potential region of one or more drusen (see FIGS. 16A and 16B).

Defined Region as drusen: In the edge detection process, all edges are detected including those associated with vessels and noise. As a result, potential regions may include a vessel or other pathology such as, a micro-aneurism. In this step, any potential region which is not one or more drusen may be eliminated. Since drusen are brighter than the background of the image 300, the ratio between potential regions and the background may be calculated. A ratio greater than 1 is defined as one or more drusen otherwise it is removed as a wrongly identified region. The value of background intensity is defined by the mean intensity plus two standard deviations of the intensity of the image except for the potential regions. Then the pixel intensity of the potential regions may be divided by the background intensity. This output may then be combined with the intensity ratio based drusen detection to obtain the final output. FIG. 17 shows an example of the drusen detected by our proposed method.

### Extract Parameters of the Changed Drusen

After registration 112 and drusen detection 120, the changes in one or more drusen may be quantified 140. The quantification may include a number of computations from two drusen-segmented images 300i, 300ii. Let's assume that the first visit is the reference image (300i) and the second visit is the subsequent image (300ii), which is transformed using image registration. Then using the overlap of the mask of the field of view of those two images, the shared area is found where the parameters of the changes in the one or more drusen are quantified. Then (r−m)>0 gives the disappeared area of drusen, (m−r)>0 gives the newly appeared area of drusen. The ‘AND’ operation (m&r) gives the steady area of drusen.

The following non-limiting examples illustrate the disclosure. These examples should not be construed as limiting; examples are included for the purposes of illustration only. The Examples will be understood to represent an exemplification of the disclosure.

### EXAMPLES

### Experiments and Results for Drusen Quantification

We have computed the accuracy of drusen detection and parameters of changes in one or more drusen to demonstrate the performance of method 100. We used NAT-2 Study data for evaluating the proposed method [7]. In the NAT-2 study there are 271 patients' images for five visits over two years, but some patients are missed in some visits.

An experienced grader selected 21 images from 11 patients randomly. Then the drusen was manually graded to compare to the result achieved by method 100. The results of method 100 have been compared with the method described in Bhuiyan et al., a state-of-the-art drusen segmentation algorithm, [8]. The performance shows method 100 outperforms the other method as shown in Table I. Table I shows the drusen segmentation accuracy with precision (Pre.), recall (Rec.), specificity (Spe.), sensitivity (Sen.), f1-score (F1-s) and accuracy (Acc.).

TABLE I
THE PERFORMANCE OF THE DRUSEN DETECTION ALGORITHM.
The performance of the drusen detection algorithm.

| | Pre. | Rec. | Spe. | Sen. | F1-s | Acc. |
|---|---|---|---|---|---|---|
| Bhu.a | 0.09 | 0.52 | 0.90 | 0.52 | 0.14 | 0.90 |
| Proposed.b | 0.79 | 0.73 | 0.99 | 0.73 | 0.76 | 0.99 |

Bhuiyan et al. [8],
bOur Proposed Method

Method 100 achieved an overall Pearson Correlation Coefficient of 0.94 against manually detected drusen which are far better than Bhuiyan et al. [8] for the tracking parameters of the drusen and reported in Table II.

TABLE II
THE PEARSON CORRELATION COEFFICIENT VALUES FOR
EACH TRACKING PARAMETER.
The Pearson Correlation Coefficient values for each tracking parameter.

| Parameters | Bhuiyan et al. | Proposed |
|---|---|---|
| Newly appeared | 0.11 | 0.87 |
| Disappeared | 0.21 | 0.99 |
| Steady | −0.24 | 0.93 |
| Overall | 0.19 | 0.94 |

The ocular disease or condition may include any ocular disease or condition and may for example include one or more of age-related macular degeneration (AMD); glaucoma; angioid streaks; retinitis pigmentosa; glaucoma and/or neoplasm of the choroid, cranial nerves, retinal or eyeball.

One embodiment of a personal device 200 suitable for use in the present disclosure is shown in FIGS. 18 and 19. In the embodiment shown personal device 200 includes a computer module 201 including input devices such as a keyboard 202, a mouse pointer device 203, a scanner 216, an external hard drive, and a microphone 280; and output devices including a printer 215, a display device 214 and loudspeakers 217. In some embodiments video display 214 may include a touchscreen.

A Modulator-Demodulator (Modem) transceiver device 216 may be used by the computer module 201 for communicating to and from a communications network 220 via a connection 221. The network 220 may be a wide-area network (WAN), such as the Internet, a cellular telecommunications network, or a private WAN. Through the network 220, computer module 201 may be connected to other similar personal devices, e.g., computer 290 or server computers 291. Where the network 220 is a telephone line, the modem 216 may be a traditional “dial-up” modem. Alternatively, where the network 220 is a high capacity (e.g.: cable) connection, the modem 216 may be a broadband modem. A wireless modem may also be used for wireless connection to network 210.

The computer module 201 typically includes at least one processor 205, and a memory 206 for example formed from semiconductor random access memory (RAM) and semiconductor read only memory (ROM). The computer module 201 also includes a number of input/output (I/O) interfaces including: an audio-video interface 207 that couples to the video display 204, loudspeakers 217 and microphone 280; an I/O interface 208 for the keyboard 202, mouse 203, scanner 216 and external hard drive; and an interface 208 for the external modem 216 and printer 215. In some implementations, modem 216 may be incorporated within the computer module 201, for example within the interface 208. The computer module 201 also has a local network interface 222 which, via a connection, 223, permits coupling of the personal device 200 to a local computer network 212, known as a Local Area Network (LAN).

As also illustrated, the local network 222 may also couple to the wide network 220 via a connection 224, which would typically include a so-called “firewall” device or device of similar functionality. The interface 201 may be formed by an Ethernet circuit card, a Bluetooth wireless arrangement or an IEEE 802.11 wireless arrangement or other suitable interface.

The I/O interfaces 208 and 203 may afford either or both of serial and parallel connectivity, the former typically being implemented according to the Universal Serial Bus (USB) standards and having corresponding USB connectors (not illustrated).

Storage devices 209 are provided and typically include a hard disk drive (HDD) 200. Other storage devices such as, an external HD 217, a disk drive (not shown) and a magnetic tape drive (not shown) may also be used. An optical disk drive 202 is typically provided to act as a non-volatile source of data. Portable memory devices, such as optical disks (e.g.: CD-ROM, DVD, Blu-Ray Disc), USB-RAM, external hard drives and floppy disks for example, may be used as appropriate sources of data to the personal device 200. Another source of data to personal device 200 is provided by the at least one server computer 291 through network 210.

The components 205 to 203 of the computer module 201 typically communicate via an interconnected bus 204 in a manner that results in a conventional mode of operation of personal device 200. In the embodiment shown in FIGS. 18 and 19, processor 205 is coupled to system bus 204 through connections 208. Similarly, memory 206 and optical disk drive 202 are coupled to the system bus 204 by connections, e.g., networks. Examples of personal devices 200 on which the described arrangements can be practiced include IBM-PC's and compatibles, Sun Sparc stations, Apple computers; smart phones; tablet computers or like a device including a computer module like computer module 201. It is to be understood that when personal device 200 includes a smart phone or a tablet computer, display device 204 may include a touchscreen and other input and output devices may not be included such as, mouse pointer device 203; keyboard 202; scanner 216; and printer 205.

FIG. 19 is a detailed schematic block diagram of processor 205 and a memory 234. The memory 234 represents a logical aggregation of all the memory modules, including the storage device 209 and semiconductor memory 206, which can be accessed by the computer module 201 in FIG. 18.

The methods of the disclosure may be implemented using personal device 200 wherein the methods may be implemented as one or more software application programs 233 executable within computer module 201. In particular, the steps of the methods of the disclosure may be effected by instructions 231 in the software carried out within the computer module 201

The software instructions 231 may be formed as one or more code modules, each for performing one or more particular tasks. The program 233 may also be divided into two separate parts, in which a first part and the corresponding code modules performs the method of the disclosure and a second part and the corresponding code modules manage a graphical user interface between the first part and the user.

The program 233 may be stored in a computer readable medium, including in a storage device of a type described herein. The software is loaded into the personal device 200 from the computer readable medium or through network 210 or 213, and then executed by personal device 200. In one example the program 233 is stored on storage medium 215 that is read by optical disk drive 202. Program 233 is typically stored in the HDD 200 or the memory 206.

A computer readable medium having such programs 233 or computer program recorded on it is a computer program product. The use of the computer program product in the personal device 200 preferably effects a device or apparatus for implementing the methods of the disclosure.

In some instances, the software application programs 233 may be supplied to the user encoded on one or more disk storage medium 215 such as a CD-ROM, DVD or Blu-Ray disc, and read via the corresponding drive 202, or alternatively may be read by the user from the networks 210 or 212. Still further, the software can also be loaded into the personal device 200 from other computer readable media. Computer readable storage media refers to any non-transitory tangible storage medium that provides recorded instructions and/or data to the computer module 201 or personal device 200 for execution and/or processing. Examples of such storage media include floppy disks, magnetic tape, CD-ROM, DVD, Blu-ray Disc, a hard disk drive, a ROM or integrated circuit, USB memory, a magneto-optical disk, or a computer readable card such as a PCMCIA card and the like, whether or not such devices are internal or external of the computer module 201. Examples of transitory or non-tangible computer readable transmission media that may also participate in the provision of software application programs 233, instructions 231 and/or data to the computer module 201 include radio or infra-red transmission channels as well as a network connection 210, 213, 334, to another computer or networked device, e.g., computer 290, 291 and the Internet or an Intranet including email transmissions and information recorded on Websites and the like.

The second part of the application programs 233 and the corresponding code modules mentioned above may be executed to implement one or more graphical user interfaces (GUIs) to be rendered or otherwise represented upon display 204. Through manipulation of, typically, keyboard 202, mouse 203 and/or screen 204 when including a touchscreen, a user of personal device 200 and the methods of the disclosure may manipulate the interface in a functionally adaptable manner to provide controlling commands and/or input to the applications associated with the GUI(s). Other forms of functionally adaptable user interfaces may also be implemented, such as an audio interface utilizing speech prompts output via loudspeakers 207 and user voice commands input via microphone 280. The manipulations including mouse clicks, screen touches, speech prompts and/or user voice commands may be transmitted via network 210 or 212.

When the computer module 201 is initially powered up, a power-on self-test (POST) program 250 may execute. The POST program 250 is typically stored in a ROM 249 of the semiconductor memory 206. A hardware device such as the ROM 249 is sometimes referred to as firmware. The POST program 250 examines hardware within the computer module 201 to ensure proper functioning, and typically checks processor 205, memory 234 (209, 206), and a basic input-output systems software (BIOS) module 251, also typically stored in ROM 249, for correct operation. Once the POST program 250 has run successfully, BIOS 251 activates hard disk drive 200. Activation of hard disk drive 200 causes a bootstrap loader program 252 that is resident on hard disk drive 200 to execute via processor 205. This loads an operating system 253 into RAM memory 206 upon which operating system 253 commences operation. Operating system 253 is a system level application, executable by processor 205, to fulfill various high level functions, including processor management, memory management, device management, storage management, software application interface, and generic user interface.

Operating system 253 manages memory 234 (209, 206) in order to ensure that each process or application running on computer module 201 has sufficient memory in which to execute without colliding with memory allocated to another process. Furthermore, the different types of memory available in the personal device 200 must be used properly so that each process can run effectively. Accordingly, the aggregated memory 234 is not intended to illustrate how particular segments of memory are allocated, but rather to provide a general view of the memory accessible by computer module 201 and how such is used.

Processor 205 includes a number of functional modules including a control unit 239, an arithmetic logic unit (ALU) 240, and a local or internal memory 248, sometimes called a cache memory. The cache memory 248 typically includes a number of storage registers 244, 245, 246 in a register section storing data 247. One or more internal busses 241 functionally interconnect these functional modules. The processor 205 typically also has one or more interfaces 242 for communicating with external devices via the system bus 204, using a connection 208. The memory 234 is connected to the bus 204 by connection 209.

Application program 233 includes a sequence of instructions 231 that may include conditional branch and loop instructions. Program 233 may also include data 232 which is used in execution of the program 233. The instructions 231 and the data 232 are stored in memory locations 218, 219, 230 and 235, 236, 237, respectively. Depending upon the relative size of the instructions 231 and the memory locations 218-230, a particular instruction may be stored in a single memory location as depicted by the instruction shown in the memory location 230. Alternately, an instruction may be segmented into a number of parts each of which is stored in a separate memory location, as depicted by the instruction segments shown in the memory locations 218 and 219.

In general, processor 205 is given a set of instructions 243 which are executed therein. The processor 205 then waits for a subsequent input, to which processor 205 reacts by executing another set of instructions. Each input may be provided from one or more of a number of sources, including data generated by one or more of the input devices 202, 203, or 204 when including a touchscreen, data received from an external source across one of the networks 210, 212, data retrieved from one of the storage devices 206, 209 or data retrieved from a storage medium 215 inserted into the corresponding reader 202. The execution of a set of the instructions may in some cases result in output of data. Execution may also involve storing data or variables to the memory 234.

The disclosed arrangements use input variables 254 that are stored in the memory 234 in corresponding memory locations 255, 256, 257, 258. The described arrangements produce output variables 261 that are stored in the memory 234 in corresponding memory locations 262, 263, 264, 265. Intermediate variables 268 may be stored in memory locations 259, 260, 266 and 267.

The register section 244, 245, 246, the arithmetic logic unit (ALU) 240, and the control unit 239 of the processor 205 work together to perform sequences of micro-operations needed to perform “fetch, decode, and execute” cycles for every instruction in the instruction set making up the program 233. Each fetch, decode, and execute cycle includes: (a) a fetch operation, which fetches or reads an instruction 231 from memory location 218, 219, 230; (b) a decode operation in which control unit 239 determines which instruction has been fetched; and (c) an execute operation in which the control unit 239 and/or the ALU 240 execute the instruction.

Thereafter, a further fetch, decode, and execute cycle for the next instruction may be executed. Similarly, a store cycle may be performed by which the control unit 239 stores or writes a value to a memory location.

Each step or sub-process in the methods of the disclosure may be associated with one or more segments of the program 233, and may be performed by register section 244-246, the ALU 240, and the control unit 239 in the processor 205 working together to perform the fetch, decode, and execute cycles for every instruction in the instruction set for the noted segments of program 233.

One or more other computers 290 may be connected to the communications network 210 as seen in FIG. 18. Each such computer 290 may have a similar configuration to the computer module 201 and corresponding peripherals.

One or more other server computer 291 may be connected to the communications network 210. These server computers 291 response to requests from the personal device or other server computers to provide information.

Method 100 may alternatively be implemented in dedicated hardware such as one or more integrated circuits performing the functions or sub functions of the described methods. Such dedicated hardware may include graphic processors, digital signal processors, or one or more microprocessors and associated memories.

It is understood that in order to practice method 100 as described above, it is not necessary that the processors and/or the memories of the processing machine be physically located in the same geographical place. That is, each of the processors and the memories used in the disclosure may be located in geographically distinct locations and connected so as to communicate in any suitable manner. Additionally, it will be understood that each of the processor and/or the memory may be composed of different physical pieces of equipment. Accordingly, it is not necessary that a processor be one single piece of equipment in one location and that the memory be another single piece of equipment in another location. That is, it is contemplated that the processor may be two pieces of equipment in two different physical locations. The two distinct pieces of equipment may be connected in any suitable manner. Additionally, the memory may include two or more portions of memory in two or more physical locations.

To explain further, processing as described above is performed by various components and various memories. It will be understood, however, that the processing performed by two distinct components as described above may, in accordance with a further embodiment of the disclosure be performed by a single component. Further, the processing performed by one distinct component as described above may be performed by two distinct components. In a similar manner, the memory storage performed by two distinct memory portions as described above may, in accordance with a further embodiment of the disclosure, be performed by a single memory portion. Further, the memory storage performed by one distinct memory portion as described above may be performed by two memory portions.

Further, various technologies may be used to provide communication between the various processors and/or memories, as well as to allow the processors and/or the memories of the disclosure to communicate with any other entity, i.e., so as to obtain further instructions or to access and use remote memory stores, for example. Such technologies used to provide such communication might include a network, the Internet, Intranet, Extranet, LAN, an Ethernet, a telecommunications network (e.g., a cellular or wireless network) or any client server system that provides communication, for example. Such communications technologies may use any suitable protocol such as TCP/IP, UDP, or OSI, for example.

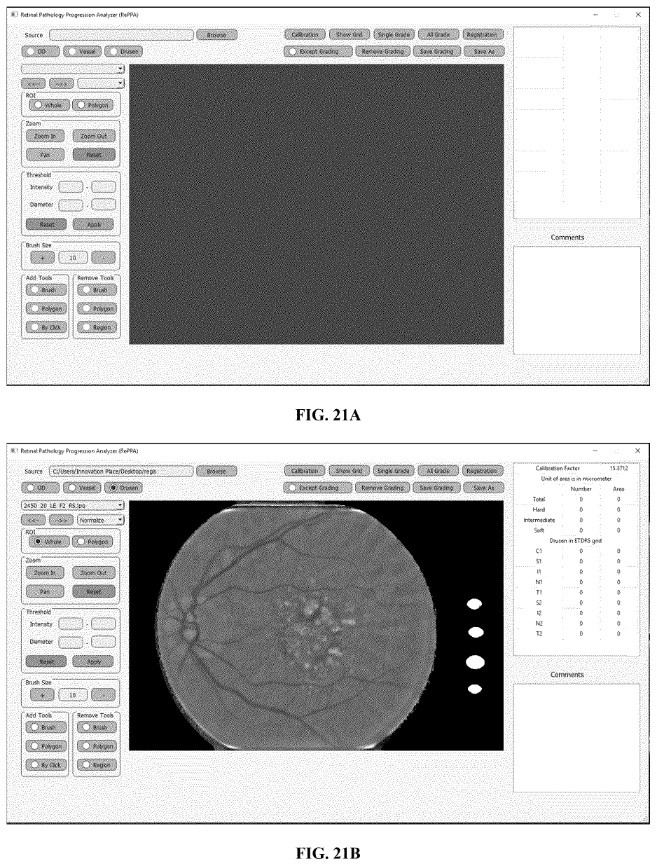

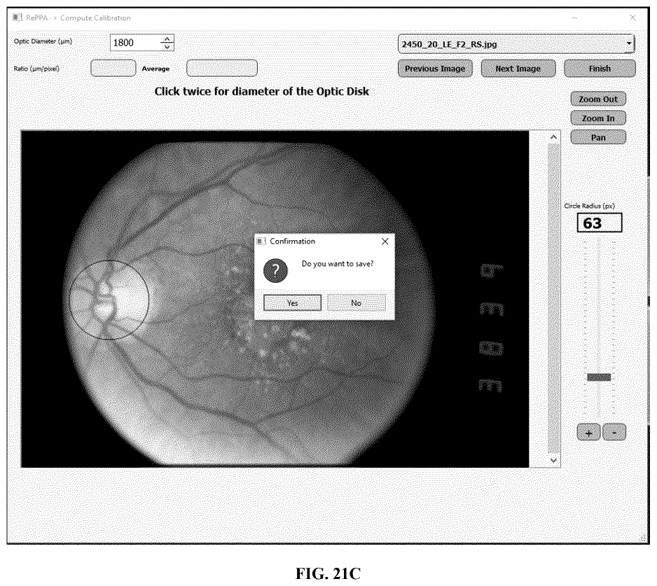

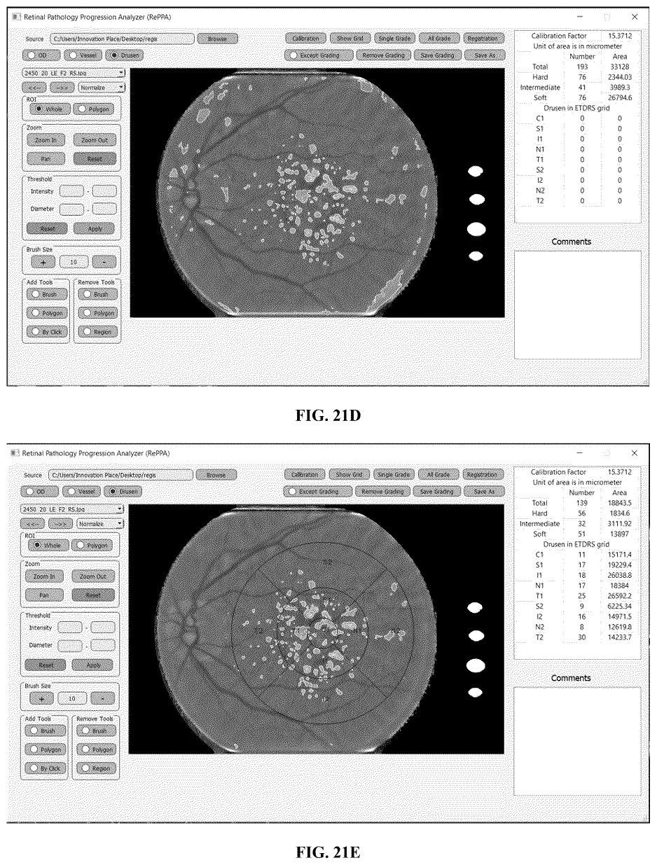

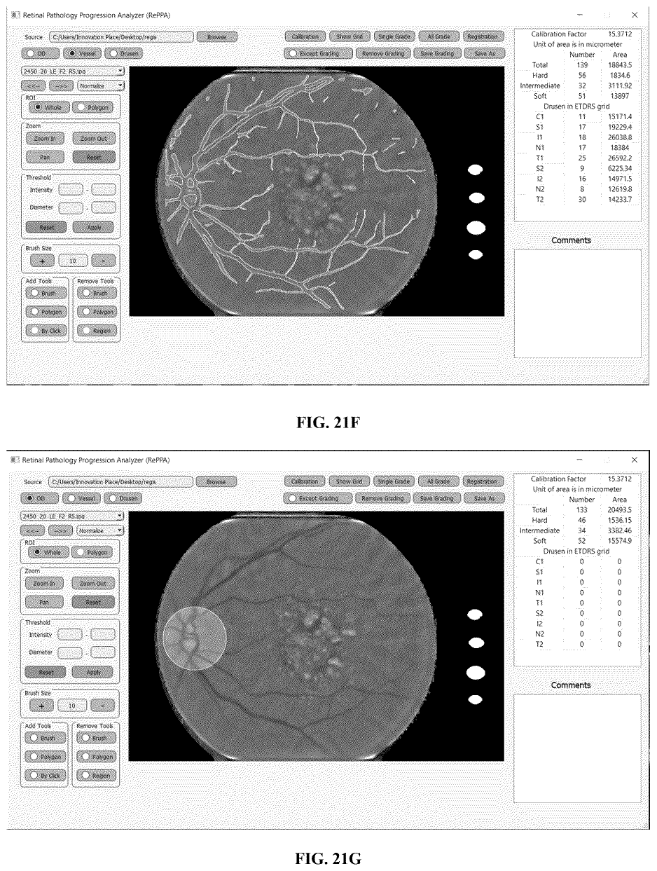

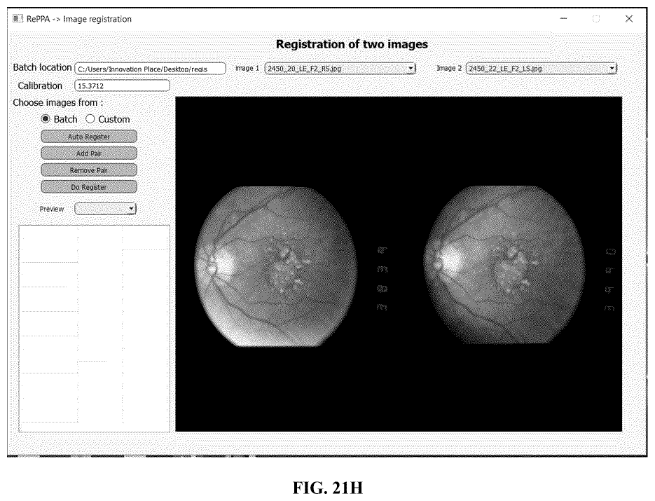

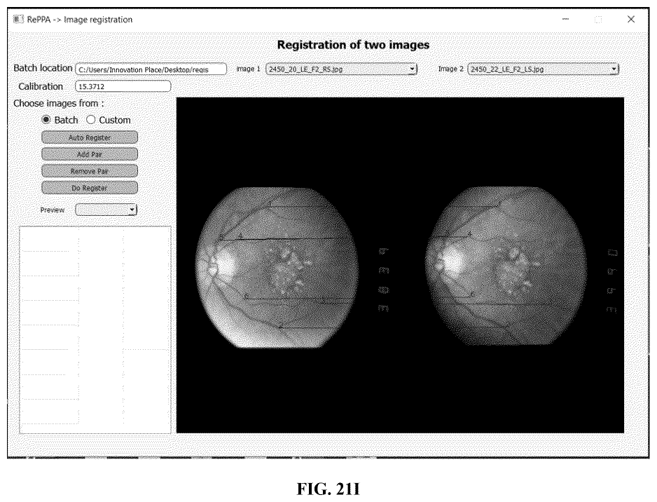

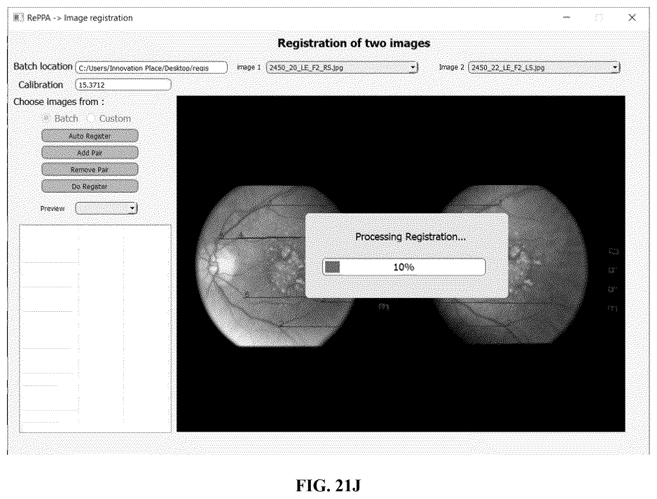

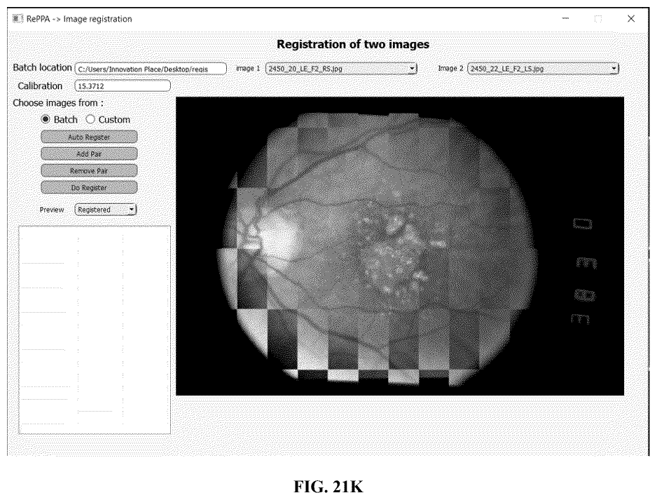

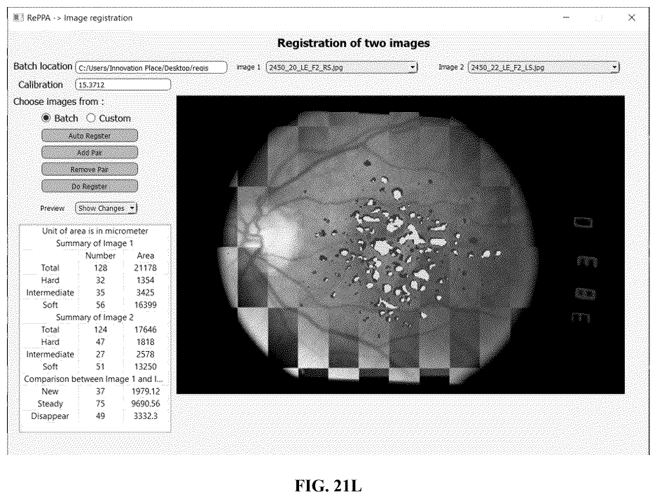

The images along with subject encrypted ID will be uploaded into the system from the user/doctor's computer. The images and data will be saved in the cloud based system (FIG. 20). The software interface (FIG. 21A) with the option of opening left/right eye image upload, zooming option, preprocessing, segmentation, editing the segmented pathology regions, zooming, selecting various shaped region (circular, rectangular or other shapes) for automatic processing, selecting multiple images for registration, and change detection in the pathology area will enable the disease progression analysis and reporting option (FIG. 21A to FIG. 21L).

Significantly, method 100 may also be implemented in a telemedicine platform. Method 100 also allows high speed relay with minimal delay through standard telemedicine and network portals along with the transfer between distant sites of patient medical records, medical images 300 and output data from medical devices.

### Algorithms

Below are exemplary algorithms, Algorithm 1 and Algorithm 2.

### 21. Algorithm 1: FindingPotentialCorrespondingBranchPoints

Input: features1, features2

### Output: MP

i. Find the alignment between each pair of the branch points
ii. Find the metric (σ_{ang}, M_{ang}, σ_{wid}, M_{wid})
iii. Δ_{ang}=30; Δ_{wid}=1.5; δ_{ang}=5; δ_{wid}=0.1; δ_{wid}=0.1; ΔMP=1;
iv. MP=[ ]
v. while δ_{ang}<Δ_{ang} and δ_{wid}<Δ_{wid}
   a. δ_{ang}=δ_{ang}+1
   b. δ_{wid}=δ_{wid}+0.05
   c. δ_{MP}=Δ_{MP}
   d. iϵFor each branch points in features1
   e. jϵFor each branch points in features2
      i. If σ_{ang} (I,j)<δ_{ang} and σ_{wid} (I,j)<δwid
         1. Match=[i,j]
         2. P_{MP}=find all pair where σ_{ang} is between [M_{ang} (I,j)−δ_{ang} and M_{ang} (I,j)+δ_{ang}] and σ_{wid} is between [M_{wid} (I,j)−δ_{wid} and M_{wid} (I,j)+δ_{wid}]
         3. kϵFor each pair of the P_{MP}
         a. If ratio of the distance and difference of angle between any pair in Match and P_{MP}(k) maintain the condition of finding the P_{MP} (above condition) then add P_{MP}(k) in Match.
         4. If length (Match)>δ_{MP}
         a. Add Match in MP as a separate list.
         b. If length (Match)>Δ_{MP}
         i. Δ_{MP}=length (Match)
vi. remove the duplicate list in MP
   22. Algorithm 2: Detection of the potential region of drusen from seed points

### PotentialRegionDrusen

Input seedPoints, img
Output potentialRegion
i. Detect edges of the image (img) using Canny Edge Detection Algorithm
ii. For each seed points in seedPoints
   Find nearest edge pixels in 0 to 360 angle
   Find largest line of connected nearest edge pixels
   If the largest line does not cover more than 0 to 90 degree angle then Remove the seed point
   Else
   All nearest edge pixels from the grass node
   The end points of the longest lines are the start and end nodes of the graph Remove all edge pixels of longest line from nodes
   Compute edge weight using Euclidian distance of pixels (nodes) except direct connection between start and end nodes
   Apply Dijkstra's shortest path algorithm
   If no shortest path found
   Then
   Remove this seed point
   Else
   Apply convex hull on the edge pixels belonging to the shortest path and largest line
   Add the region found from convex hull as potential region of drusen (potentialRegion)

The work is supported by NIH SBIR Grant #2R44EY031202-04A1.

In this specification, the terms “comprises”, “comprising” or similar terms are intended to mean a non-exclusive inclusion, such that an apparatus that comprises a list of elements does not include those elements solely but may well include other elements not listed.

Throughout the specification the aim has been to describe the disclosure without limiting the disclosure to any one embodiment or specific collection of features. Persons skilled in the relevant art may realize variations from the specific embodiments that will nonetheless fall within the scope of the disclosure.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PLI, scripting languages, Visual Basic, metalanguages which themselves specify programs, and all first, second, third, fourth, fifth, or further generation computer languages. Also included are database and other data schemas, and any other meta-languages. No distinction is made between languages that are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above are also intended to be within the scope of the disclosure.

### REFERENCES

[1] U. T. V Nguyen, A. Bhuiyan, L. A. F. Park, and K. Ramamohanarao, “An effective retinal blood vessel segmentation method using multi-scale line detection,” Pattern Recognit., vol. 46, no. 3, pp. 703-715, 2013.
[2] O. Ronneberger, P. Fischer, and T. Brox, “U-Net: Convolutional Networks for Biomedical Image Segmentation,” Miccai, pp. 234-241, 2015.
[3] H. Fu, Y. Xu, D. W. K. Wong, and J. Liu, “Retinal vessel segmentation via deep learning network and fully-connected conditional random fields,” 2016 IEEE 13th Int. Symp. Biomed. Imaging, pp. 698-701, 2016.
[4] Daniele Cortinovis, “Retina blood vessel segmentation with a convolutional neural network,” 2016. [Online]. Available: https://github.com/orobix/retina-unet. [Accessed: 6 Feb. 2018].
[5] C. Hernandez-Matas, X. Zabulis, A. Triantafyllou, P. Anyfanti, S. Douma, and A. A. Argyros, “FIRE: fundus image registration dataset,” J. Model. Ophthalmol., vol. 1, no. 4, pp. 16-28, 2017.
[6] A. Bhuiyan, R. Kawasaki, T. Y. Wong, and R. Kotagiri, “A new and efficient method for automatic optic disc detection using geometrical features,” in World Congress on Medical physics and Biomedical Engineering, 2009, vol. 25, no. 4, pp. 1131-1134.
[7] N. M. Pumariega, R. T. Smith, M. A. Sohrab, V. Letien, and E. H. Souied, “A prospective study of reticular macular disease,” Ophthalmology, vol. 118, no. 8, pp. 1619-1625, 2011.
[8] A. Bhuiyan, C. Karmakar, D. Xiao, K. Ramamohanarao, and Y. Kanagasingam, “Drusen quantification for early identification of age related macular degeneration (AMD) using color fundus imaging,” Conf. Proc. . . . Annu. Int. Conf. IEEE Eng. Med. Biol. Soc. IEEE Eng. Med. Biol. Soc. Annu. Conf., vol. 2013, pp. 7392-7395, 2013.

What is claimed is:

1. A system for detecting one or more pathology or an indication of one or more pathology, disease or condition progression, the system comprising:
a processor configured to:
register two or more retinal images, the two or more images including images obtained at differing times;
detect one or more pathology or indication of one or more pathology, disease or condition in the registered two or more images;
locate the detected one or more pathology or indication of one or more pathology, disease or condition in a plurality of the two or more images; and
quantify the one or more pathology or indication of the one or more pathology or indication in a plurality of the two or more images to thereby detect the one or more pathology or indication of the one or more pathology, disease or condition progression.

2. A method for detecting one or more pathology or an indication of one or more pathology, disease, or condition progression, the method comprising:
registering two or more retinal images, the two or more images including images obtained at differing times;
detecting one or more pathology or indication of one or more pathology, disease or condition in the registered two or more images;
locating the detected one or more pathology or indication of one or more pathology, disease or condition in a plurality of the two or more images; and
quantifying the one or more pathology or indication of the one or more pathology or indication in a plurality of the two or more images to thereby detect the one or more pathology or indication of the one or more pathology, disease or condition progression.

3. A computer program product for detecting one or more pathology or an indication of one or more pathology, disease or condition progression, the computer program product comprising:
a computer usable medium and computer readable program code embodied on said computer usable medium for displaying data, the computer readable code including:
computer readable program code devices (i) configured to cause the computer to register two or more retinal images, the two or more images including images obtained at differing times;
computer readable program code devices (ii) configured to cause the computer to detect one or more pathology or indication of one or more pathology, disease or condition in the registered two or more images;
computer readable program code devices (iii) configured to cause the computer to locate the detected one or more pathology or indication of one or more pathology, disease or condition in a plurality of the two or more images; and
computer readable program code devices (iv) configured to cause the computer to quantify the one or more pathology or indication of the one or more pathology or indication in a plurality of the two or more images to thereby detect the one or more pathology or indication of the one or more pathology, disease or condition progression.

4. The system of claim 1, further comprising providing one or more parameters of the quantified one or more pathology or indication of one or more pathology, disease or condition.

5. The system of claim 1, wherein the registering includes an automatic registration or a semi-automatic registration.

6. The system of claim 1, wherein the image registration includes one or more of retinal blood vessel segmentation; vessel centerline computation; detecting and extracting parameters of branch points of the vessels; finding potential matched branch point lists among the two or more images; determining a best registration method; and applying a transformation matrix.

7. The system of claim 1, wherein the one or more pathology or indication of one or more pathology, disease or condition in the registered two or more images may include at least one of one or more drusen or one or more bright lesions in the retina.

8. The system of claim 7, wherein at least one of the one or more drusen or one or more bright lesions is detected using a segmentation and quantification method.

9. The system of claim 1, wherein registration includes identifying a same or identical reference location of a retina in a plurality of the two or more by images.

10. The system of claim 1, wherein a same pathological feature or indication of pathology, disease or condition is found on a plurality of the two or more images.

11. The system of claim 1, wherein one or more parameter may be at least one of extracted or provided that may be used in a report.

12. The system of claim 7, wherein at least one of the one or more drusen or one or one or more bright lesions may be tracked in a longitudinal study to observe disease or condition progression or a predisposition thereto.

13. The system of claim 1, wherein each of the two or more images may include a retinal image.

14. The system of claim 1, wherein each of the two or more images may be captured locally or received from one or more local or remote source.

15. The system of claim 1, wherein at least one of drusen or bright lesions may be tracked.

16. The system of claim 1, wherein registration includes four modules.

17. The system of claim 1, wherein at least one of one or more non-drusen features or non-bright lesion may be removed.

18. The system of claim, 7 wherein detection of at least one of the one or more drusen or bright lesion includes combining two or more detection methods.

19. The system of claim 1, wherein quantification includes a number of computations from two drusen-segmented images.

20. The system of claim 1, wherein the one or more pathology or an indication of one or more pathology, disease or condition progression includes an ocular pathology or an indication of one or more ocular pathology, disease or condition progression.
